(19) United States
(12) Patent Application Publication
(10) Pub. No.: US 2014/0058291 A1
(43) Pub. Date: Feb. 27, 2014

(54) System and Method for Measuring a Body Force

(71) Applicant: ABFIT Products, LLC, Richardson, TX (US)

(72) Inventors: Warren Abraham Wartell, Dallas, TX (US); Barry Stewart Brown, Springdale, AR (US); Gene Lee Armstrong, Richardson, TX (US)

(73) Assignee: ABFIT Products, LLC, Richardson, TX (US)

(21) Appl. No.: 13/962,594

(22) Filed: Aug. 8, 2013

Related U.S. Application Data
(60) Provisional application No. 61/692,308, filed on Aug. 23, 2012.

(51) Int. Cl. A61B 5/22

(52) U.S. Cl. CPC A61B 5/224
USPC 600/595

(57) ABSTRACT

An apparatus includes a back plate having an upper surface and a lower surface, a body contact device connected to the back plate, and a measurement device connected to the body contact device. The body contact device resists movement in a direction substantially perpendicular to the upper surface of the back plate while the measurement device measures a force applied to the body contact device.

This application claims the benefit of U.S. Provisional Application No. 61/692,308, filed on Aug. 23, 2012, entitled “Apparatus for the Measurement of Abdominal Strength,” which application is incorporated herein by reference in its entirety.

### TECHNICAL FIELD

The present invention relates generally to measurement devices. In particular, embodiments the present invention relate to a system and method for measuring a body force.

### BACKGROUND

Recently, interest in muscular strength and body composition has drastically increased. The emergence of clinical protocols for testing muscular strength has provided clinicians with a variety of techniques to measure muscular strength in an individual. These protocols are often used to determine a variety of risk factors based on an individual's musculature, posture, and body composition.

Abdominal strength has become particularly important, as abdominal musculature has been linked to the incidence and prevalence of low back health. Strong abdominal muscles, such as the rectus abdominis and obliques, help normalize the lordotic curve of the spine, thereby helping to prevent spinal injuries. Accordingly, the strength of the rectus abdominis and associated muscles may provide prophylactic value with regard to back health. As a result, abdominal strengthening exercises have been prescribed to prevent and rehabilitate low back injury.

While many different techniques have been developed for testing abdominal strength, significant shortcomings remain. The most common procedure involves time and repetition count of a sit up maneuver. In some cases, however, these procedures may not provide a desired level of accuracy or safety. For example, sit up type tests and other protocol using repetition in a timed segment is by definition a measure of endurance, not strength. Moreover, testing protocols may not be designed to minimize arching of the back or lumbar extension, which can exacerbate lumbar lordosis. Additional protocols may be needed to provide valid, repeatable, and safe conditions for testing abdominal strength in individuals.

### SUMMARY OF THE INVENTION

In accordance with an embodiment of the present invention, an apparatus includes a back plate having an upper surface and a lower surface, a body contact device connected to the back plate, and a measurement device connected to the body contact device. The body contact device is configured to resist movement in a direction substantially perpendicular to the upper surface of the back plate. The measurement device is configured to measure a force applied to the body contact device.

In accordance with an alternative embodiment of the present invention, an abdominal strength measurement system includes a metal housing, a load cell, and a body strap. The metal housing has a top face, a bottom face, and two side faces connecting the top face and the bottom face. The load cell is arranged within an interior of the metal housing. The body strap is connected to the load cell and is configured to resist movement in a direction substantially perpendicular to the top face of the metal housing.

In accordance with yet another embodiment of the present invention, a method for measuring strength is presented. An apparatus including a back plate having an upper surface and a lower surface is provided. A force exerted against a body contact device connected to the back plate is measured. The force is exerted in a direction perpendicular to the upper surface of the back plate. A strength measurement is determined from the force exerted against the body contact device.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 is an illustration of a Cartesian coordinate reference to the human body;

FIGS. 2A-2D are illustrations of cross-sectional views of a torso with a force measurement device;

FIGS. 3A-3C are illustrations of a chest oriented device for measuring abdominal strength;

FIGS. 4A-4D are illustrations of a back oriented device for measuring abdominal and torsional strength;

FIG. 5 is an illustration of a back oriented device for measuring abdominal strength;

FIG. 6 is an illustration of a self-adjusting back oriented device for measuring abdominal strength;

FIGS. 7A-7C are illustrations of a detachable back oriented device for measuring abdominal strength;

FIGS. 8A and 8B are illustrations of a bench with a back oriented device for measuring abdominal strength;

FIG. 9 is an illustration of a plan view of a device for measuring abdominal strength;

FIGS. 10A-10C are illustrations of cross-sectional views of a device for measuring abdominal strength;

FIG. 11 is an illustration of a perspective view of a device for measuring abdominal strength;

FIG. 12 is an illustration of the interior of a device for measuring abdominal strength; and

FIGS. 13A and 13B are illustrations of structural components within a device for measuring abdominal strength.

Corresponding numerals and symbols in the different figures generally refer to corresponding parts unless otherwise indicated. The figures are drawn to clearly illustrate the relevant aspects of the embodiments and are not necessarily drawn to scale.

### DETAILED DESCRIPTION OF ILLUSTRATIVE EMBODIMENTS

The making and using of various embodiments are discussed in detail below. It should be appreciated, however, that the present invention provides many applicable inventive concepts that can be embodied in a wide variety of specific contexts. The specific embodiments discussed are merely illustrative of specific ways to make and use the invention and do not limit the scope of the invention.

Abdominal musculature has been linked to the incidence and prevalence of low back health with a strong rectus abdominis and associated muscles providing prophylactic value. Strong abdominal muscles help normalize the lordotic curve by producing a more posterior pelvic tilt, thereby preventing impingement of the sensory nerve as it passes through the intervertebral foramen. As a result, abdominal strengthening exercise is used to prevent and rehabilitate low back injury, reverse muscle imbalances, and improve posture, among others.

To test abdominal strength, many different techniques have been developed, the most prevalent of which is the timed sit up test. Unfortunately, most procedures have been severely criticized from both a safety and validity viewpoint. The major issue surrounding the safety and validity of tests to assess abdominal strength has been concern with the definition of strength. Sit up type tests and other protocol using repetition in a timed segment is by definition a measure of endurance. Strength, on the other hand, is a single maximal force over a short time period.

While muscle endurance reveals an individual's ability to contract a muscle over a period of time until fatigue, an endurance measurement may not provide the prophylactic value of a strength measurement. With regard to abdominal strength, muscular endurance does necessary correlate with increased back health. Additionally, these sit up tests may compromise safety by causing an individual to arch her back, exacerbating lumbar lordosis.

During currently used abdominal assessments, the validity of the measurement also may be compromised due to a variety of factors. For instance, any movement beyond 45° to the horizontal generates activity in the hip flexors (i.e., the psoas, iliacus, and rectus femoris), invoking use of non-abdominal musculature. Movement beyond 45° to the horizontal also causes the beginning of a gravitational assist. As another example, holding of the lower extremities (typically feet and/or ankles) invites a major contribution from the hip flexor muscles during the sit up procedure. Each of these factors decreases the validity of the test results, while putting the individual at risk of injury.

Thus, various embodiments provide a measurement apparatus in which muscular abdominal activity is maximized, hyperextension is substantially reduced, and the rectus abdominis and auxiliary lumbar spinal flexors (e.g., external oblique abdominis) are active throughout the testing sequence. In an embodiment, the rectus abdominis and associated spinal flexors are isolated, thus eliminating the use of the hip flexors and spinal extensors. This isolation is achieved by maintaining an angle of force substantially 45° to the horizontal, or less, and without holding the individual's ankles or feet. With the use of an illustrative embodiment, isolation and measurement of the rectus abdominis is achieved. Accordingly, this strength measurement may be used to correlate an individual with various health-related risk factors.

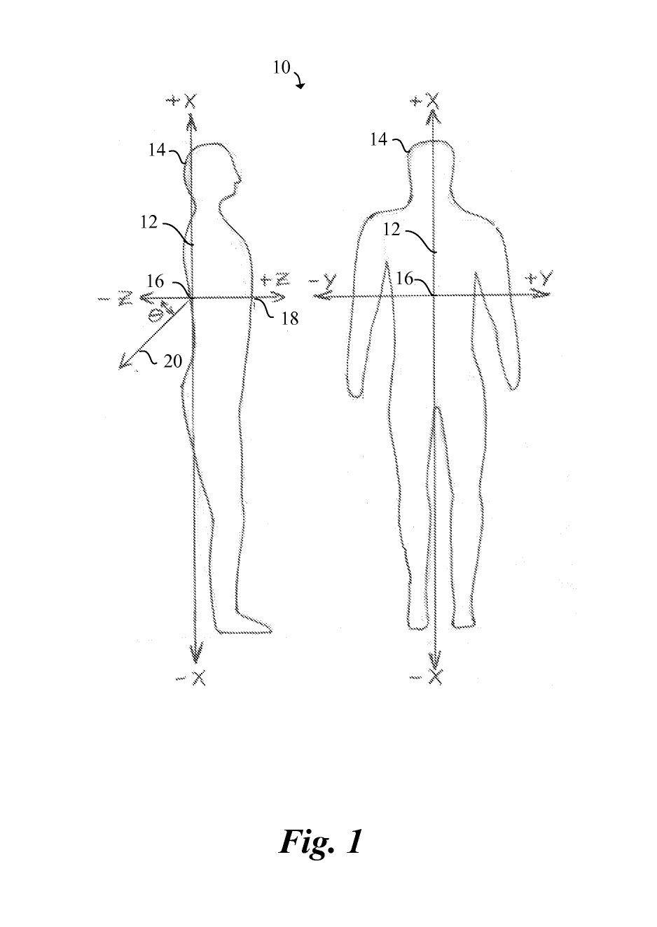

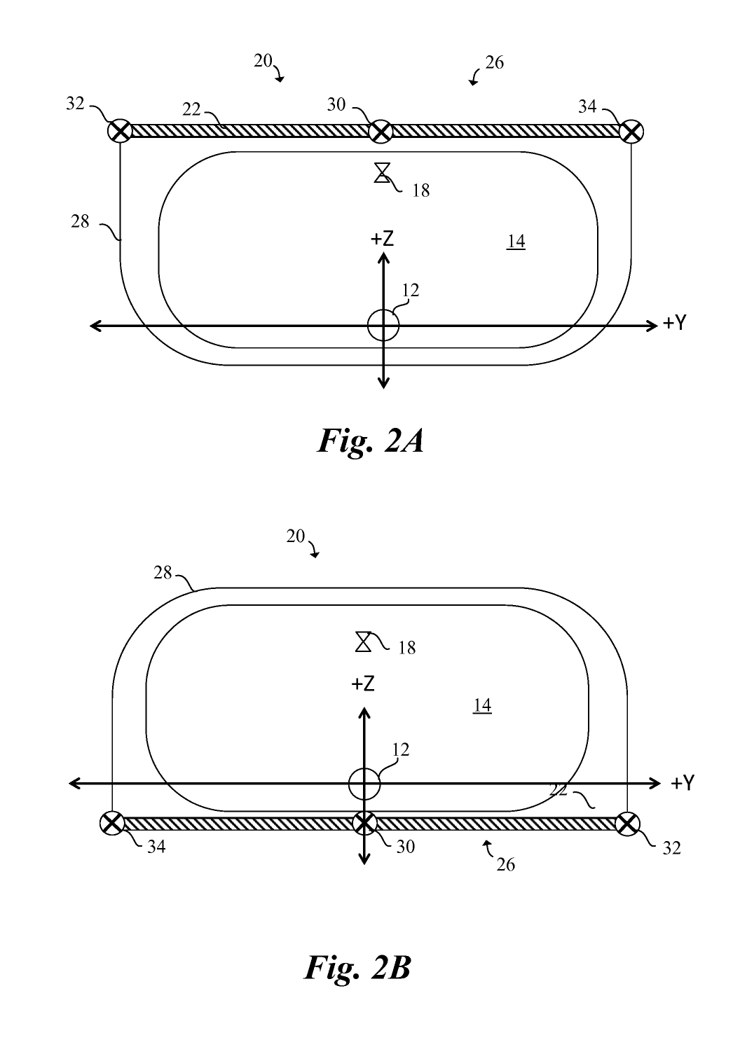

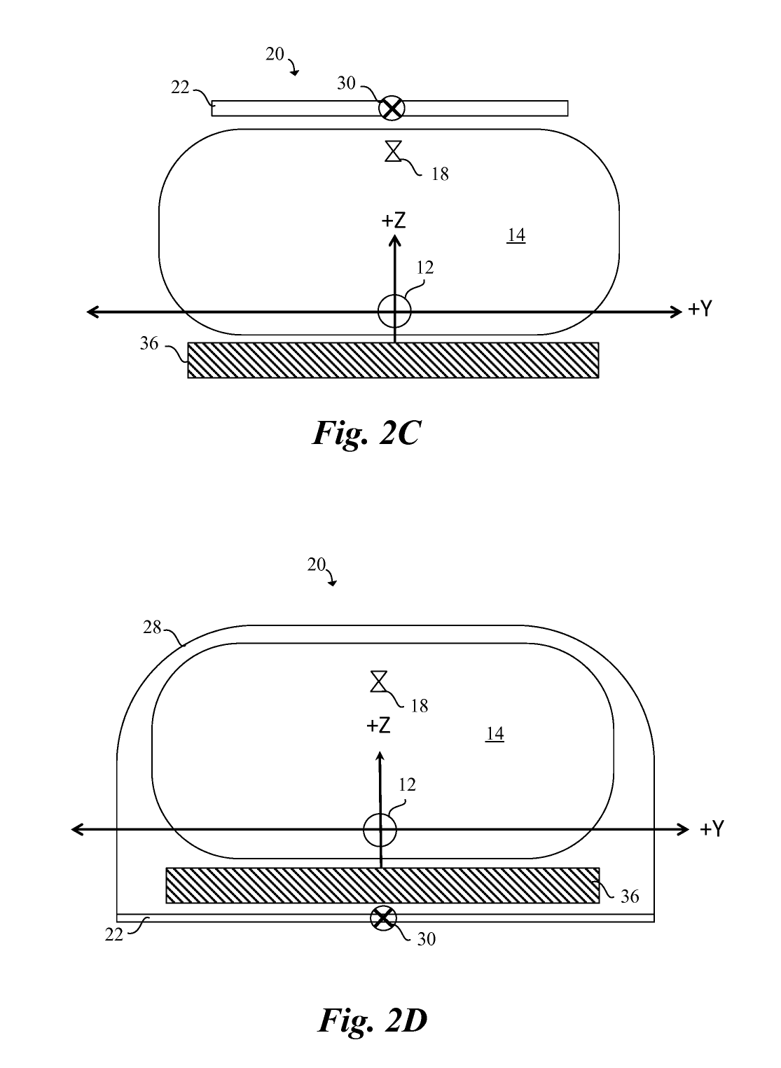

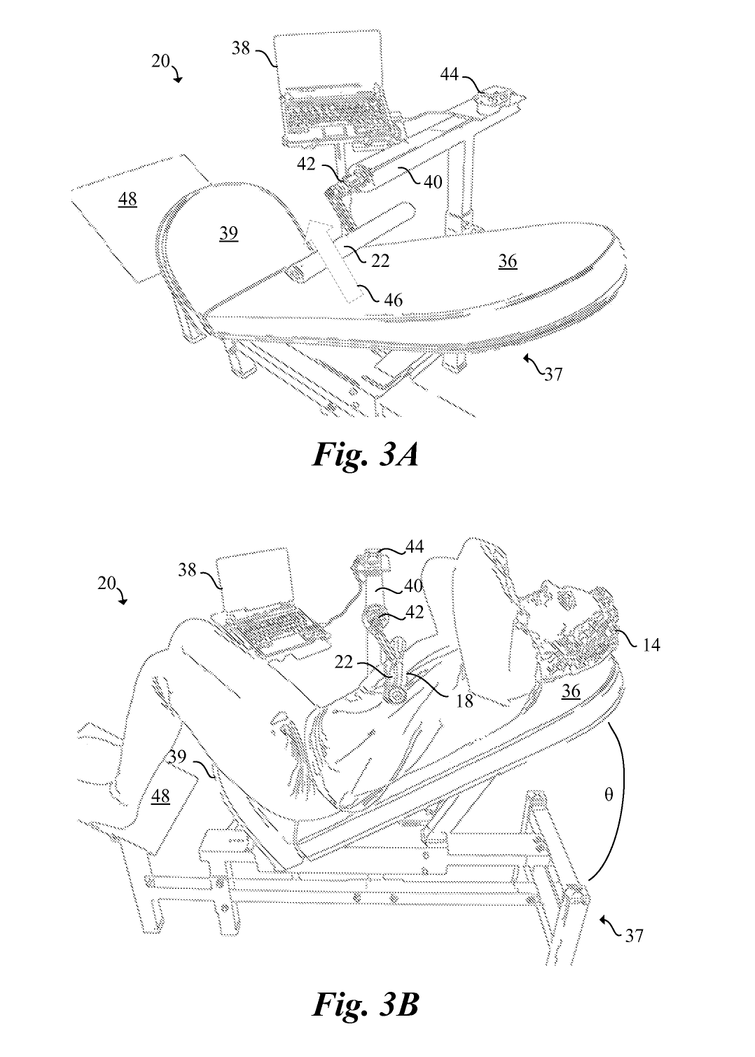

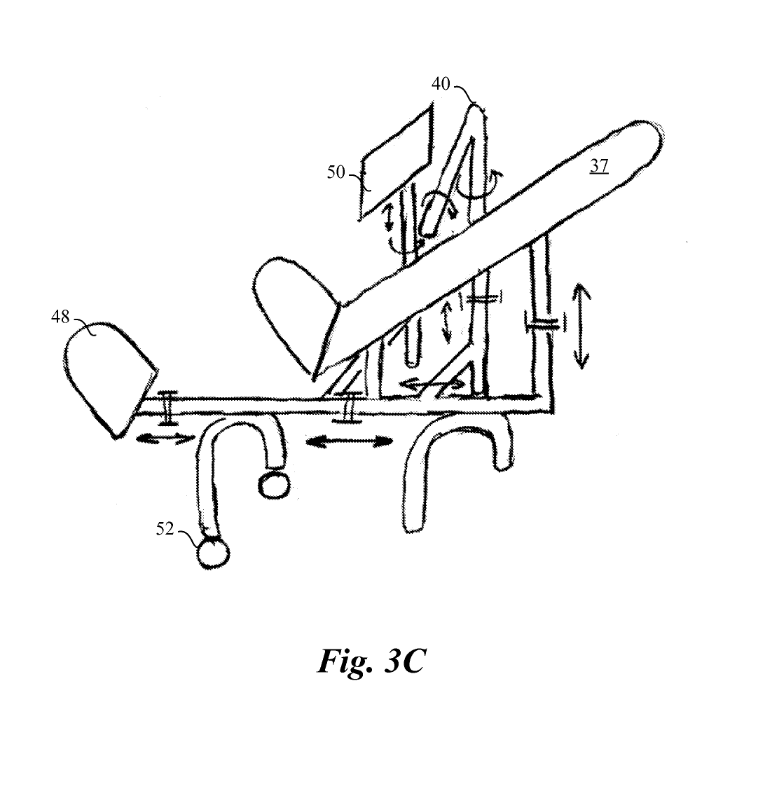

A Cartesian coordinate reference to the human body is described using FIG. 1, various layouts of the apparatus are described using FIGS. 2-13, and a method of operating the apparatus is described with reference to FIG. 3B.

Referring to FIG. 1, a coordinate system 10 for the description of force sensor configurations is shown. In coordinate system 10, the x-axis is coincident with a spinal column 12 of a subject 14 under test. The origin of the axis is an intersection 16, where spinal column 12 intersects with a normal projection to a xiphoid process 18 of subject 14. An angle from the horizontal is quantified by theta (θ) and a vector 20 represents the gravitational source vector of the system. Coordinate system 10 is used herein with reference to FIGS. 2-6.

FIG. 2, which includes FIGS. 2A-2D, illustrates a cross-sectional view of a portion of the torso of subject 14. Particularly, FIG. 2A shows a cross-sectional view of a torso with a measurement system 20 arranged on the torso. In this view, measurement system 20 is a crest oriented multi-function force measurement device.

As illustrated, a body contact device 22 with a measurement device 24 is arranged along the y-axis, perpendicular to spinal column 12 of subject 14. In particular, body contact device 22 is arranged along xiphoid process 18 of subject 14.

In this example, measurement device 24 includes a plurality of force sensors 26. Force sensors 26 include sensors 30, 32, and 34. In this embodiment, force sensors 26 are strain gauge load cells. Other forms of force measurement, such as spring loaded linear potentiometers or optical interferometers may be substituted for load cells without loss of utility.

Body contact device 22 may be a bar, a tube, a plate, a strap, or some other type of device attached to force sensors 26. Body contact device 22 is designed to distribute the force applied by subject 14 along the y-axis for the purpose of subject comfort. In an embodiment, the dimensions of body contact device 22 are minimal in the x-direction such that body contact device 22 covers substantially all of xiphoid process 18. For example, the width of body contact device 22 may be less than one and one-half inch. The width of body contact device 22 may be small in order to minimize force spread over the anterior torso region of subject 14.

In this embodiment, body contact device 22 is adjustable such that it may move to accommodate different sizes of test subjects. For instance, body contact device 22 may be adjusted to move along the x-axis and/or y-axis to place force sensors 26 on body contact device 22 in the desired position relative to xiphoid process 18 of subject 14.

Body contact device 22 is configured such that it substantially resists the movement of subject 14 in the +z-direction. When subject 14 moves, force sensors 26 measure force exerted against body contact device 22 in the +z-direction by direct contact with body contact device 22 by subject 14. In other words, as subject 14 performs a sit up and presses xiphoid process 18 against the body contact device 22, force sensors 26 take a measurement of the force exerted by subject 14.

With reference to the illustrative embodiment, a sit up activity is a motion where subject 14 is attempting to sit up by bending at the waist through a contraction in the rectus abdominis and auxiliary muscles of the anterior torso. In a pure sit up motion, the majority of force is exerted against body contact device 22 using the rectus abdominis muscles. Therefore, this force measurement corresponds to an abdominal strength measurement of subject 14.

Variations in body position or muscle imbalances may cause variations in sit up movement, depending on the subject. Measurement system 20 may be designed to take these differences into account. For example, more force sensors 26 may be placed along body contact device 22 to detect imbalances in muscle contraction between the left and right side of subject 14.

In some embodiments, force sensors 26 may measure force exerted against body contact device 22 in the −z-direction through a strap 28 wrapped around subject 14 and secured to body contact device 22. In this embodiment, strap 28 is also adjustable to aid in subject comfort. Strap 28 may be arranged to elicit a force of about five pounds during the sit up motion to prevent slippage of the chest assembly during the sit up motion. The strap 28 may be configured to weigh about one pound in one example. Other weights and forces may also be realized, depending on the particular implementation of the strap assembly. Strap 28 is part of body contact device 22 in these examples.

To measure a force in the +z-direction, as would be experienced in a sit up activity, or in the −z-direction, as experienced in a backward arch of the back, a single sensor 30 may be used. Additional force sensors 26, such as force sensors 32 and 34, arranged along body contact device 22 also may be used to increase the accuracy and versatility of the force measurement. For example, if a measurement of torsional strength (i.e. twisting of the torso) is desired as well as z-axis force measurements, sensors 32, 34 may be used. More than three sensors also may be arranged along body contact device 22, depending on the particular implementation.

In FIG. 2B, measurement system 20 is oriented in the opposite direction from FIG. 2A. Particularly, body contact device 22 is arranged posterior to spinal column 12 of subject 14, while strap 28 crosses the torso of subject 14. Subject 14 may be placed in measurement system 20 such that substantially the center of strap 28 rests on xiphoid process 18. Measurement system 20 in this Figure is a back oriented multi-function force measurement device.

In this example, the back portion of the measurement system 20 may be set at an angle from the horizontal such that the abdominal muscles exert the majority of the force against body contact device 22. Accordingly, the abdominal muscles are substantially isolated in this embodiment and other muscles, such as the hip flexors, do not exert force on body contact device 22.

As depicted, subject 14 exerts a force against strap 28 in the +z-direction, which is measured by force sensors 26 as a function of the force exerted on body contact device 22 from strap 28. Force sensors 26 may also provide a direct measurement of force exerted on body contact device 22 in the −z-direction as subject 14 arches his back or extends his spine. A torsional force measurement also may be taken using this embodiment by sensors 32, 34.

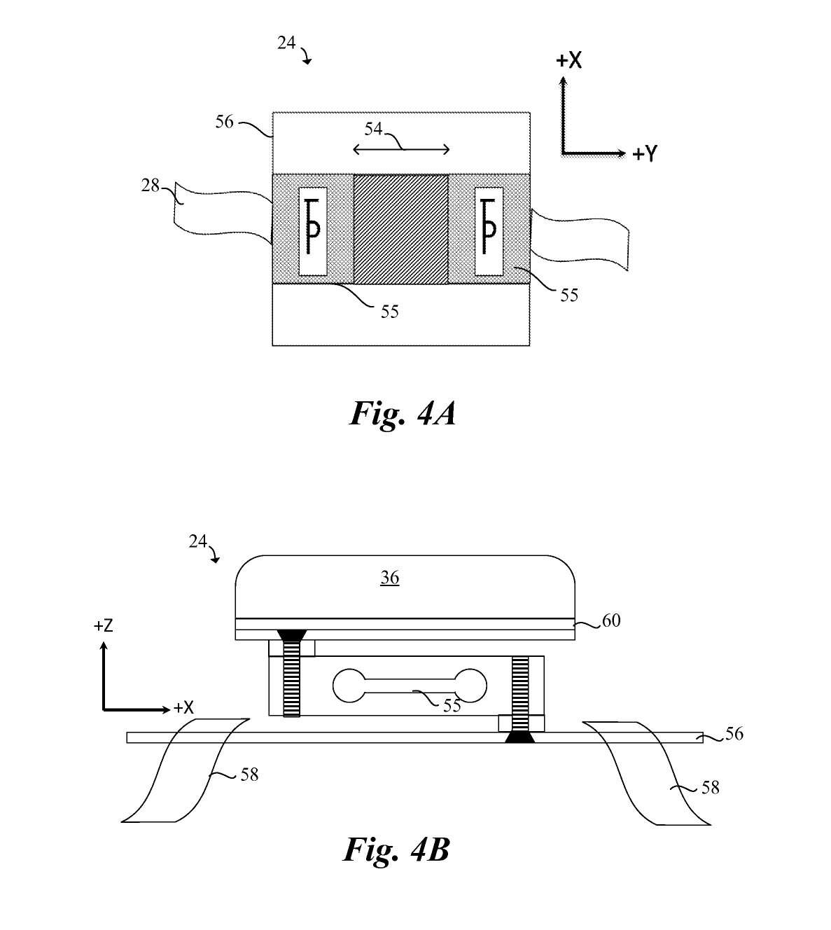

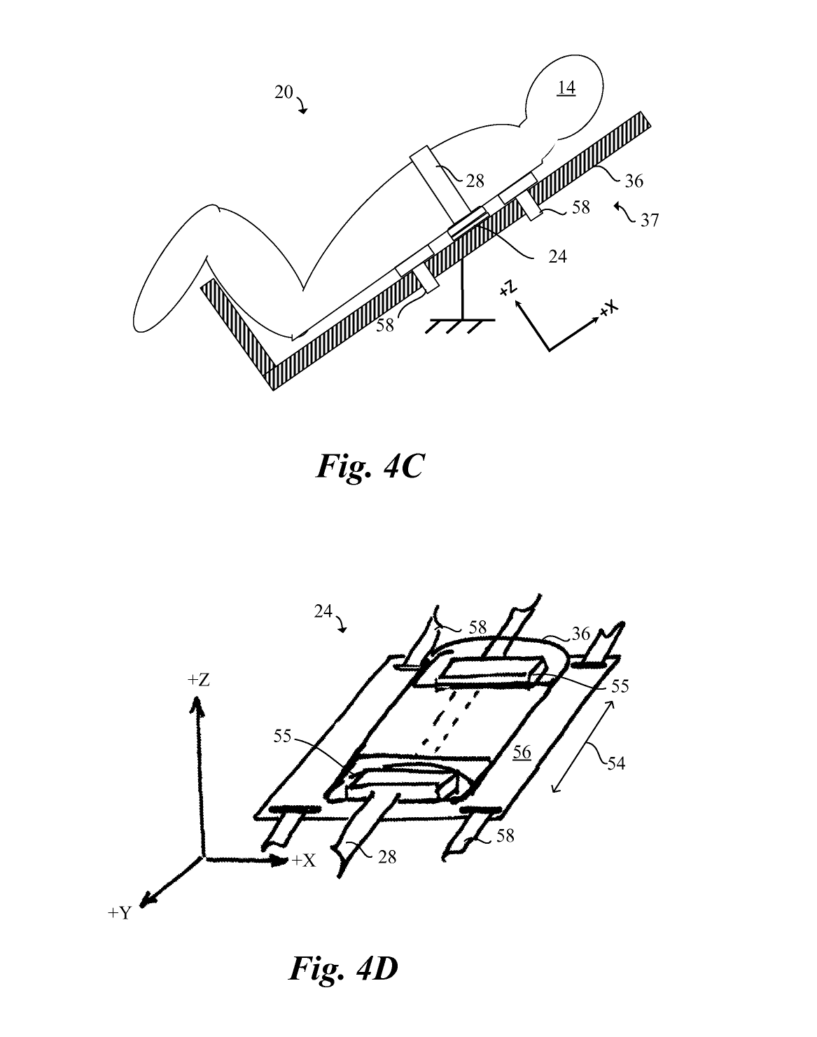

FIG. 2C shows subject 14 arranged on a back plate 36 with chest oriented measurement system 20 positioned on xiphoid process 18. In this example, back plate 36 may be an exercise bench, an exam table, or some other suitable surface that remains substantially in place during movement of subject 14. In other words, as subject 14 performs a sit up activity to exert force against body contact device 22, back plate 36 should remain stationary. Alternatively, back plate 36 may be part of a mobile measurement system 20 that may be secured to another solid surface, as described with reference to FIG. 4.

As depicted in this embodiment, measurement system 20 measures abdominal strength in the +z-direction. During a sit up test, body contact device 22 is rigidly connected to back plate 36 in some fashion. For example, measurement system 20 with body contact device 22 may be connected to back plate 36 using fasteners, such as bench belts, or may be immovably connected to back plate 36. When body contact device 22 is immovably connected to back plate 36, body contact device 22 may be welded to back plate 36, secured with screws, or coupled to back plate 36 by other components in measurement system 20.

In FIG. 2D, strap 28 has been added to measurement system 20 with back plate 36. Measurement system 20 in this embodiment is a back oriented measurement device, but does not measure force exerted in the −z-direction. Instead, body contact device 22 is arranged below a lower surface of back plate 36.

As depicted, sensor 30 measures force exerted against body contact device 22 in the +z-direction as transmitted through strap 28 by subject 14 while lying on back plate 36. In this embodiment, back plate 36 and body contact device 22 are rigidly connected during subject measurement.

FIG. 3, which includes FIGS. 3A-3C, shows measurement system 20 with data storage and display device 38. In this embodiment, body contact device 22 is a padded bar, while back plate 36 is a padded backrest of an adjustable bench 37. Adjustable bench 37 also includes seat 39.

As illustrated, measurement system 20 includes a rigidly attached arm 40 attached to body contact device 22. Both back plate 36 and arm 40 may be adjustable in order to accommodate differing test subject sizes. For example, back plate 36 adjusts horizontally on bench 37, while arm 40 adjusts vertically with respect to bench 37.

The horizontal adjustment of back plate 36 may be performed by rack-and-pinion, hydraulic jack, or other suitable linear motion actuator, or may be slid continuously and locked with a pin or clamp. Similarly, the vertical adjustment of arm 40 may be performed by rack-and-pinion, hydraulic jack, or other suitable linear motion actuator, or may be slid continuously and locked with a pin or clamp. In some embodiments, arm 40 may adjust horizontally. In still other embodiments, it may also be possible to lengthen or shorten body contact device 22. An operator may use these features to increase subject comfort and ensure accurate placement of the measurement system 20 relative to the subject.

At one end of arm 40 is a load cell transducer 42 which outputs an analog signal proportional to the force applied to body contact device 22. The load cell transducer signal is converted to a digital format by a data acquisition unit 44, which then transmits the digital formatted data to data storage and display device. Data storage and display device 38 may include a separate or integrated audio output device.

Data storage and display device 38 may be remote, detached, or affixed to measurement system 20. When remote to measurement system 20, data storage and display device 38 may be a desktop computer, a laptop computer, a tablet, a smartphone, or some other device and may be wirelessly connected to data acquisition unit 44.

In an embodiment, perpendicular alignment between a body contact bar axis 46 and the plane of back plate 36 may reduce torque on load cell transducer 42. In some embodiments, load cell transducer 42 may include an alignment plate mechanism to allow rotation of body contact device 22 a full 360° and to be firmly affixed at a specific angle for other incident angle measurements. This arrangement also may be used for easier load cell calibration by hanging a fixed heavy weight while load cell transducer 42 is positioned to align body contact device 22 in the desired vertical orientation.

In some embodiments, back plate 36 of bench 37 may be adjusted to various discrete angles (θ) to accommodate measurement of lumbar extension or oblique contraction. Further, back plate 36 may be adjusted to provide a profile of static force measurements over a range of specified angles.

In some embodiments, the back plate 36 may be part of a bench 37 that may include wheels, rails, hydraulics, pneumatics, or other components that allow bench 37 some mobility. When an abdominal strength test is performed, however, these features should be locked such that bench 37 is immobile.

In a standard abdominal measurement test using measurement system 20, an angle to the horizontal of back plate 36 may be selected that minimizes injury and maximizes accuracy of the test. In some embodiments, an angle of substantially 30° to the horizontal is particularly effective in reducing the potential for injury as a result of arching the lower back.

In some cases, smaller angles may be less effective because of the increased torque resulting from smaller angles. For example, angles of 0° may result in zero readings, as some subjects are unable to exert any force against body contact device 22 because of insufficient abdominal strength. On the other hand, larger angles, such as angles approximating 90°, may enlist gravitational assist, with the effects of gravity favoring those with higher body weight. Of course, the angle of back plate 36 may be adjusted to a different angle depending on the measurement protocol of the test.

In this example, measurement system 20 does not include a foot stabilizing device. As compared to other abdominal measurement devices that hold the feet in a stable position, measurement system 20 shown in this figure may result in a more accurate strength measurement in some embodiments. A foothold is eliminated from measurement system 20 because holding the feet or ankles results in a force much larger than that obtained without lower leg support, indicating that the hip flexors provide a majority of the force. In some embodiments, a footrest also may be included in bench 37. Since footrest 48 does not fix the position of the subject's legs or feet, footrest 48 does not affect the accuracy of the strength measurement.

In FIG. 3B, the apparatus is operable by measuring the force exerted by subject 14 on body contact device 22. Back plate 36 and arm 40 are adjusted such that body contact device 22 rests comfortably on xiphoid process 18 of the subject's anatomy while the subject's abdominal muscles are in a relaxed state. Alternately, body contact device 22 maybe positioned up to one inch below xiphoid process 18 without significant change in measured force.

When subject 14 is in a relaxed state, measurements are taken to establish a transducer tare for load cell transducer 42. Abdominal strength is measured, in force units, when subject 14 exerts a force between their xiphoid process 18 and body contact device 22 while executing a maximum effort sit up activity.

During the sit up, bench 37 and body contact device 22 are structurally rigid with respect to each other and therefore do not significantly deflect when subject 14 exerts a force. Next, a plurality of force measurements are periodically taken and stored for further analysis.

In some embodiments, force measurements are taken over ten second intervals. Measurement system 20 may be configured to provide measurements over longer or shorter intervals as well. In other examples, a series of three-second maximal contractions are measured with rest periods in between. Measurement system 20 measures force over the period in which force is exerted against body contact device 22 by subject 14. Other protocols may be used for both testing and training.

As the plurality of force measurements is taken, subject 14 maintains maximal contraction of the abdominal muscles, resulting in maximum output of force against body contact device 22. Abdominal strength is determined from these force measurements. A change in strength also may be determined over time. For example, a subject's initial abdominal contraction may exert significant force against body contact device 22 but may decline over time. Conversely, a subject's abdominal contraction may stay steady throughout the duration of the test. With this type of information at hand, clinicians may generate additional training, testing, or rehabilitative protocol for the subject. The decline in force over time may be used to generate a fatigue index or the percentage decline in force over a set time period.

One or more of these periodic force measurements may be displayed by data storage and display device 38 for instant feedback to subject 14. Alternatively or in addition, data storage and display device 38 may generate audible alerts or instructions to the subject before, during, and after the test. An overall strength score may be determined from these measurements.

In some embodiments, measurement system 20 may also include a processor that compares the subject's strength measurement to a known strength scale. For instance, a database with abdominal strength scores from a variety of test subjects may be stored in data storage and display device 38 or may be accessed in some other device. These scores may be classified by weight, height, age, activity level, physical health, or some other suitable factor or combination of factors.

Next, the strength measurement may be classified based on the comparison between the strength measurement and the known strength scale. As an example, a measurement protocol may define five risk categories for low back problems, including: (1) Immediate Attention; (2) Needs Improvement; (3) Moderate; (4) Above Average; (5) Good; and (6) Excellent. Based on a subject's abdominal strength score, he or she can be placed in one of these categories. Of course, other categories may be used to classify individuals based on abdominal strength.

Further, abdominal strength scores and muscle imbalance analysis may aid in prescribing the appropriate exercises for a training program. For example, due to injury, a subject's right oblique muscles may be stronger than his left oblique muscles. As a result, measurement system 20 may read slight torsional force imbalances during testing. With this information, a clinician can prescribe treatment to correct such imbalances which, if otherwise left alone, could cause further imbalance, pain, injury, permanent postural degradation, and/or other harm to the subject. As another example, corrective exercise prescribed in response to an abdominal strength test may be used to increase performance in athletes.

Strength measurement results may be organized in a variety of ways. For example, a report may be personalized and generated for the subject. The clinician may print the report or send the report to the client's mobile device, tablet, laptop, or other wireless communications device. This report may include one or more strength scores, identifying information for the subject, graphs, classification information, recommendations for improvement, a comparison of the subject to others in his or her age group and/or fitness level, among other data. This report may be stored in a database or data storage device for future use.

FIG. 3C identifies additional enhancements to measurement system 20 may aid in user comfort, ease of use or portability, and ease of operation. For example, seat 39 and/or back plate 36 of bench 37 may be raised, lowered, rotated, elongated, detached, or folded in order to accommodate subject entry into the apparatus, increase subject comfort, deviate the measurement angle, or make the apparatus more portable.

These adjustments may be performed by rack-and-pinion, hydraulic jack, or other suitable linear motion actuator, or may be slid or rotated continuously and locked with a pin or clamp. Automated adjustment may be achieved through motorized actuators driven by either stored subject preferences or manual entry.

In various embodiments, arm 40 may be raised, lowered, rotated, elongated, detached, moved horizontally or vertically to the floor plane, or folded in order to accommodate the subject entry or make the apparatus more portable. These adjustments also may be manual or automated.

As illustrated, measurement system 20 further includes a user interface table 50 to support data storage and display device 38. Measurement system 20 also includes footrest 48 and wheels 52. Much like the other elements shown in this figure, both the user interface table 50 and footrest 48 may be raised, lowered, rotated, elongated, detached, or folded, either manually or automatically. In some embodiments, wheels 52 may be replaced by rails or other mobile elements. The entire apparatus may be placed on wheels 52 in order to facilitate ease of transport.

In this example, the entire measurement system 20 may be folded such that measurement system 20 may be moved from place to place by a single person. When measurement system 20 is collapsible, the components may include light-weight structural materials such as aluminum, cast hardened plastic, fiberglass, carbon fiber, and other suitable materials.

FIG. 4, which includes 4A-4C, shows a back oriented measurement device 24 that measures both abdominal and torsional strength. In this embodiment, measurement device 24 measures strength from the back (rear) of the test subject.

As depicted, measurement device 24 includes two load cells 55 as force sensors, separated by a distance 54 and mounted on a rigid plate 56. This unit is attached to an upper surface of exercise bench 37, as shown in FIG. 4C, and a lower surface of back plate 36, as shown in FIG. 4B.

In this example, rigid plate 56 is a metal plate. In other embodiments, rigid plate 56 may be a different type of material such as plastic, cast hardened plastic, fiberglass, carbon fiber, or another suitable type of material. As shown, measurement device 24 may be 14 inches by 18 inches. Various embodiments may include measurement devices of other dimensions, additional load cells, or other modifications, depending on the functionality involved.

Measurement device 24 may be adjusted up and down the x-axis by bench belts 58 or other suitable mechanisms. Strap 28 is connected to load cells 55 and wrapped around subject 14. After being adjusted for subject comfort, strap 28 is secured from elastically expanding in circumference around subject 14.

In this embodiment, abdominal strength is measured as the sum of the two readings from each of the load cells 55 when subject 14 performs a sit up activity. Torsional strength is measured as the difference in the readings between the two load cells 55 multiplied by the distance 54, as subject 14 twists her torso.

As shown, rigid plate 56 is part of bench 37. In other embodiments, such as the embodiment shown in FIG. 4D, rigid plate 56 is not part of bench 37 and may be movably connected to bench 37 instead.

FIG. 4B shows a side view of measurement system 20 with load cells 55 arranged between back plate 36 and rigid plate 56. Layers 60 also may be present between back plate 36 and load cells 55. Example materials for layers 60 include plastic, foam, and other suitable materials. Layers 60 aid in subject comfort and protect components within the device from damage. In this example, back plate 36 also has a padded surface for subject comfort.

In FIG. 4C, measurement system 20 has been arranged on bench 37 and secured with bench belts 58. Bench belts 58 are configured such that measurement system 20 remains substantially fixed on bench 37 during testing of subject 14.

FIG. 4D shows an alternative view of measurement system 20, as described with respect to FIG. 4C. In this view, back plate 36 is shown in phantom. Two top plates are also seen in this view, one top plate arranged on the upper surface of each of load cells 55.

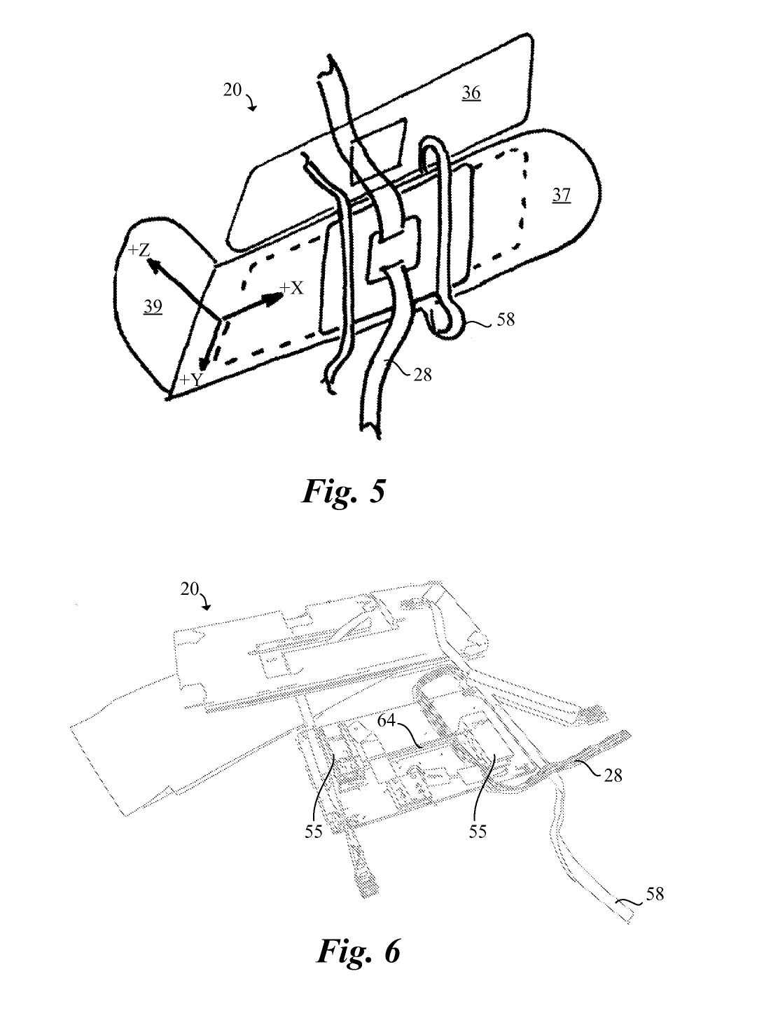

In FIG. 5, measurement system 20 includes a single load cell arranged between rigid plate 56 and strap 28 which wraps around the test subject. With the use of a single load cell, the weight of measurement system 20 may be minimized. In operation, this embodiment of measurement system 20 may be affixed to a wall, ceiling, or floor for testing of various muscle groups.

In other embodiments using a single load cell and a portable unit, measurement system 20 may be equipped with a push configuration to measure force applied to rigid plate 56. In this instance, force may be measured bi-directionally using the load cell. For example, this embodiment may be used to test a subject's posterior torso muscles such as the lumbar extensors which include the erector spinae group, multifidus, oblique extensors, latissiumus dorsi (lower region), and other small muscle groups.

FIG. 6 shows an alternative implementation for measurement system 20 which is self-adjusting to differing body dimensions. In this example, measurement system 20 includes two load cells 55 on rigid plate 56 and a rigid rod 64 that connects load cells 55 to each other.

In this embodiment, measurement system 20 is configured such that measurement device 24, either built into a bench or constructed as a detachable unit, will adjust to the dimensions of the test subject by sliding strap 28 along rod 64 until correctly positioned. When the subject performs a sit up activity, strap 28 will pull on rod 64 which is attached to load cells 55. Abdominal strength is measured as sum of the two load cell readings from the force exerted on rod 64 by subject 14.

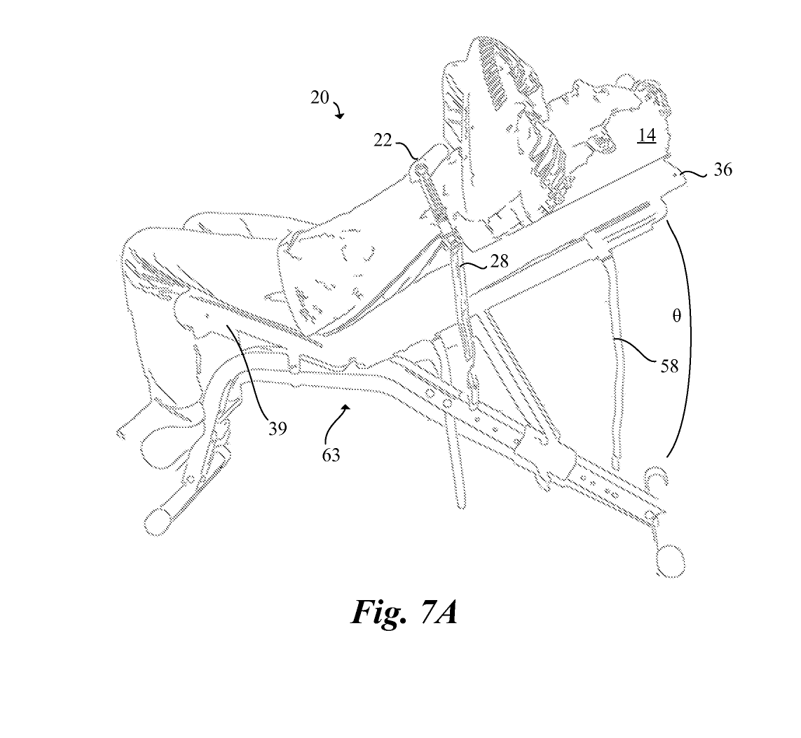

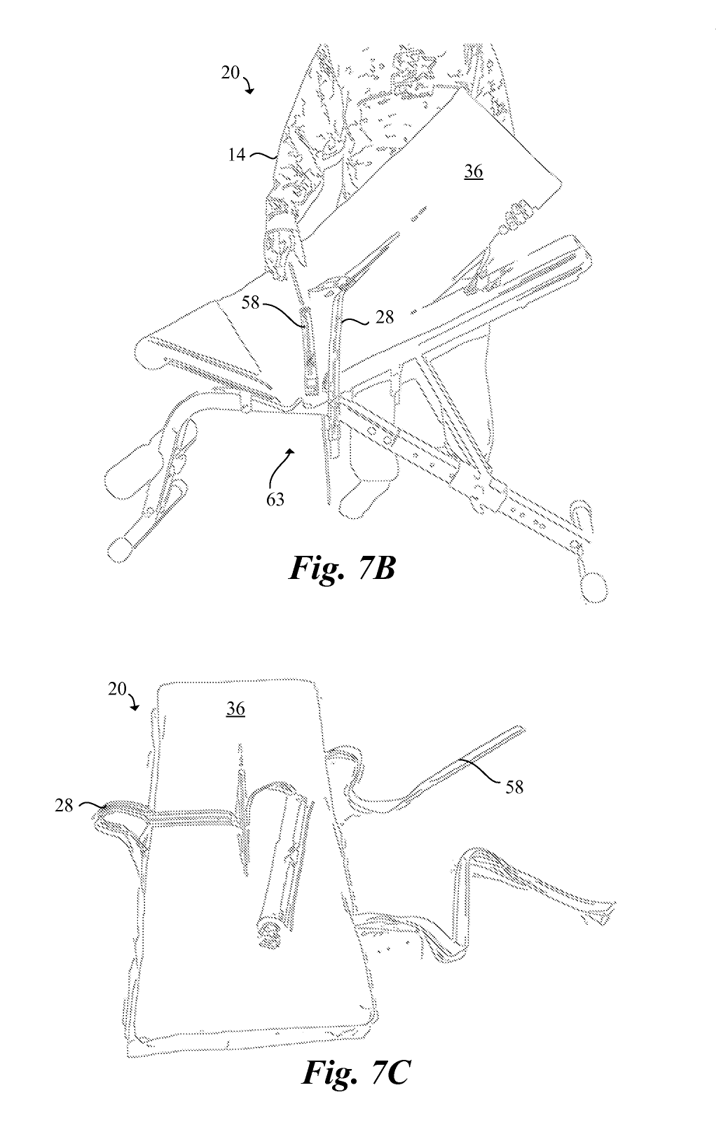

FIG. 7, which includes FIGS. 7A-7C, shows a detachable back oriented measurement system 20. In FIG. 7A, measurement system 20 is attached to a standard commercially available exercise bench 63. As discussed previously, bench attachment may be performed by fasteners such as belts, clips, sleeves, or other suitable fasteners. In this case, measurement system 20 is secured using bench belts 58. Body contact device 22 is a body strap in this embodiment.

FIG. 7B shows measurement system 20 being detached from exercise bench 63, while FIG. 7C shows a top view of the detachable measurement system 20. As can be seen from FIG. 7B, measurement system 20 is easily movable from one location to another location. Accordingly, measurement system 20 may include light-weight materials such as aluminum, cast hardened plastic, fiberglass, and carbon fiber, among others.

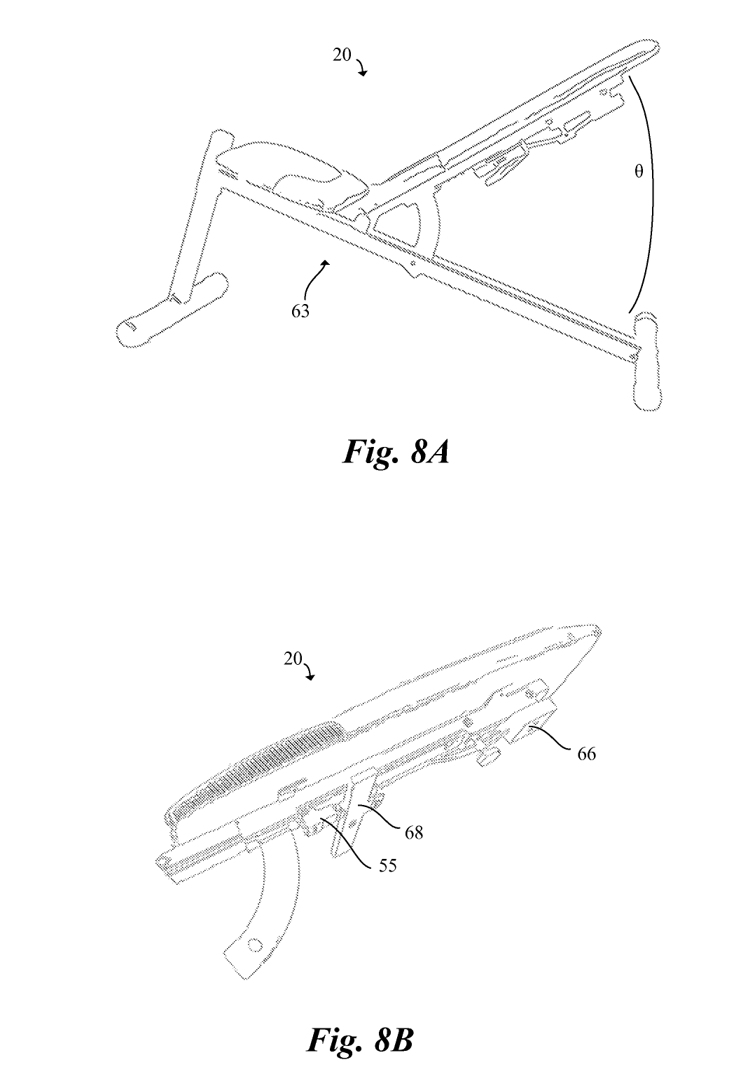

In FIG. 8, which includes FIGS. 8A and 8B, measurement system 20 is built into exercise bench 63. FIG. 8A shows measurement system 20 installed in exercise bench 63 and adjusted such that the back rest of exercise bench 63 is 30° from the horizontal. In FIG. 8B, load cell 55 is attached to a plate which may be adjusted in the x-direction and locked when properly positioned by clamp 66. A tube 68 is attached to load cell 55. Body strap 28 is threaded through tube 68 and secured around the subject who is positioned on top of exercise bench 63, as previously described.

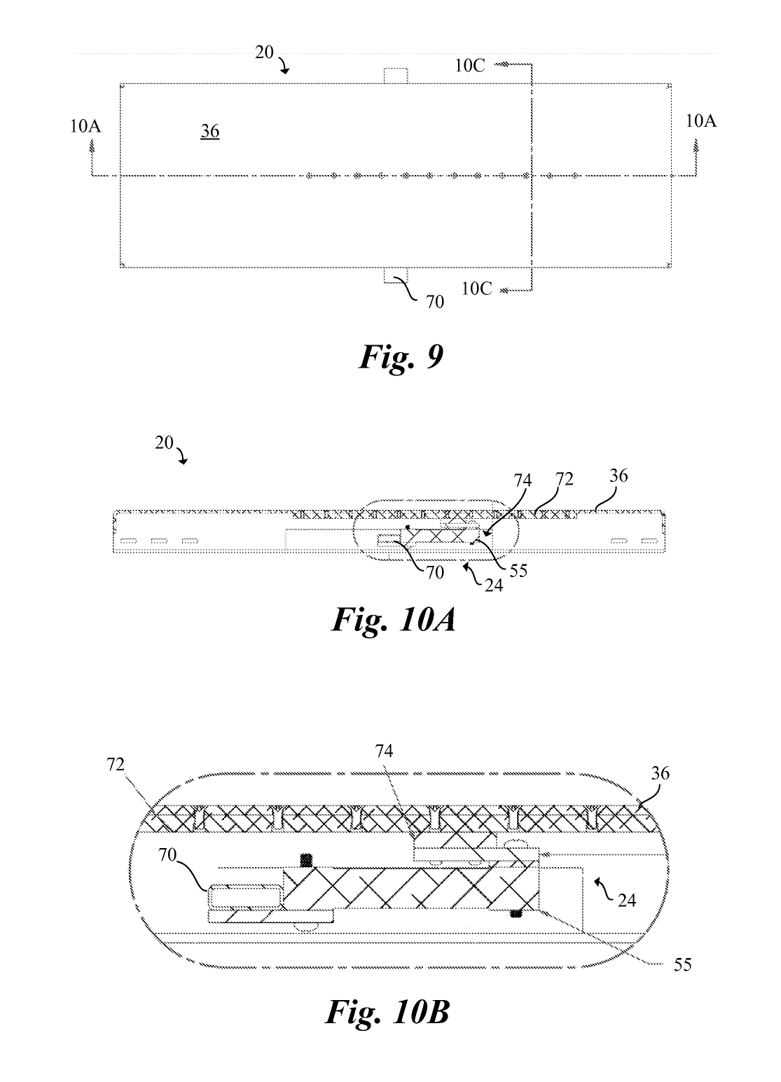

FIG. 9 shows a plan view of an alternative embodiment for the detachable measurement system 20. In this embodiment, the upper surface of back plate 36 is illustrated. Additionally, an elongate member 70 is shown protruding from a first side and a second side of back plate 36.

In this example, elongate member 70 is made of a metal material and is configured to adjust measurement device 24 (not shown) along a track connected to the lower surface of back plate 36. Elongate member 70 may include the same or different material as back plate 36. In other embodiments, both back plate 36 and elongate member 70 may be plastic, composite material, or some other suitable type of material.

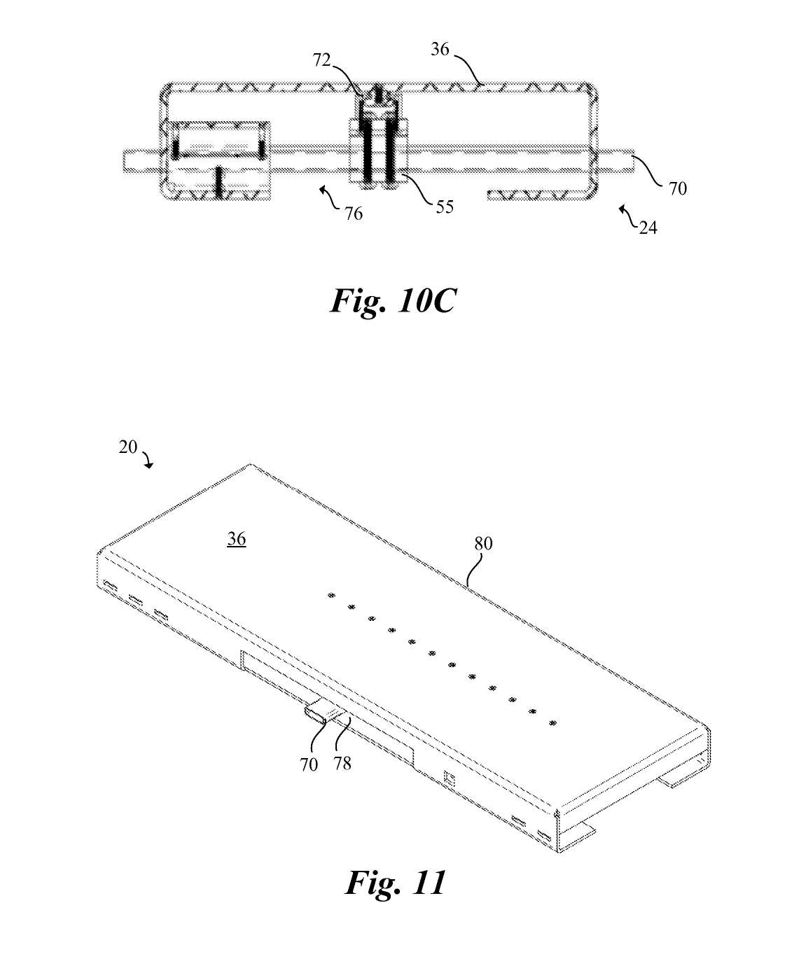

FIG. 10, which includes FIGS. 10A-10C, shows cross-sectional views of the measurement system 20 of FIG. 9. In FIG. 10A, a cross-sectional view taken along the line 10A-10A in FIG. 9 is illustrated. In this example, back plate 36 includes one contiguous sheet of metal and forms a metal housing having a top face, a bottom face, and two side faces connecting the top face and the bottom face. In alternative embodiments, the metal housing may include multiple sections that are welded, fastened, or otherwise connected together.

Measurement device 24 is arranged within the interior of the metal housing. Particularly, a slide rail 72 is arranged between measurement device 24 and the bottom face of the metal housing and is configured to couple measurement device 24 to the metal housing. In this embodiment, slide rail 72 is fixed to back plate 36 using fasteners. In other examples, slide rail 72 may be secured to back plate 36 using other means such as solder, an adhesive, or may be fabricated as part of back plate 36.

As depicted, measurement device 24 includes load cell 55, elongate member 70, and support structures 74. Support structures 74 may include beams, fasteners, welds, screws, adhesives, or other structures that secure load cell 55 and elongate member 70 to slide rail 72. A variety of materials may be used for support structures 74 such as metal, plastic, composite, wood, metal alloy, or a combination of such materials, among others.

Strap 28 (not shown) is also connected to load cell 55. In this embodiment, elongate member 70 is hollow and strap 28 is placed through elongate member 70. Measurement device 24, with load cell 55, elongate member 70, and support structures 74, is configured to move along slide rail 72 to adjust strap 28 for a test subject. When the desired setting is reached, measurement device 24 is locked into place by a clamp, rack and pinion, or some other component.

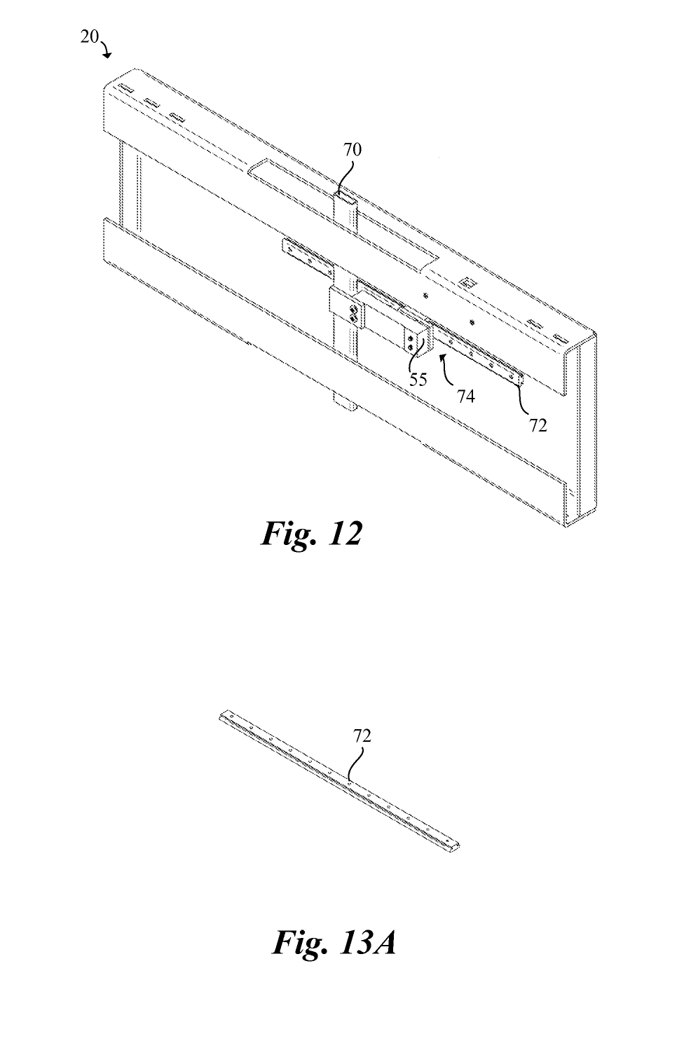

In some embodiments, measurement device 24 has the ability to move along substantially all of slide rail 72. Alternatively, measurement device 24 may be limited by the configuration of the metal housing, as shown in detail in FIGS. 11 and 12.

FIG. 10B depicts measurement device 24 more clearly. In some embodiments, support structures 74 may not be necessary or may include only one element that movably connects load cell 55 to slide rail 72.

In FIG. 10C, a cross-sectional view taken along the line 10C-10C in FIG. 9 is shown. Here, the structure of the metal housing can be seen more clearly. In particular, a gap 76 is present between two portions of the bottom surface of the metal housing. This gap 76 may decrease the weight of measurement system 20, while allowing easy reconfiguration of the components within the system. For example, an additional load cell may be added to the device without damaging or substantially altering the structural integrity of measurement device 24.

As another example, a different type of measurement device, such as a potentiometer, may be switched out for load cell 55 and accompanying support structures 74. Thus, measurement system 20 with gap 76 provides a versatile measurement system that can be reconfigured quickly and easily.

FIG. 11 shows measurement system 20 with an opening 78 in a side face of the metal housing. An opening 80 of the same size and shape is located in the side face opposite opening 78. Elongate member 70 extends through both opening 78 and opening 80.

As measurement device 24 moves along slide rail 72, elongate member 70 moves through openings 78, 80. Movement of measurement device 24 is limited by the size and shape of the openings 78, 80. Openings 78, 80 may be smaller or larger than shown in this figure, depending on the size of measurement system 20 and/or the range of movement desired.

In FIG. 12, the bottom portion of measurement system 20 is shown. As depicted, the metal housing includes one continuous piece of material. This metal housing is designed such that the bottom portion of the housing protects the electronics, load cell 55, and slide rail 72 while providing user access for easy assembly and service. Additionally, the housing is configured to have enough clearance to load cell 55 to ensure that load cell 55 and/or the fabric or foam cushioning on an underlying bench is not damaged.

The slide slits allow protected access to plug in the USB cable, and multi-positional tethering strap locations to accommodate a wide variety of existing fitness benches.

In this example, measurement device 24 has a range of adjustment on slide rail 72 and a metal housing thickness that is configured to biomechanically accommodate subjects from about 4′-about 7′ in height, weighing up to about 400 lbs. In other examples, openings 78, 80 may be modified to accommodate subjects of other heights as well.

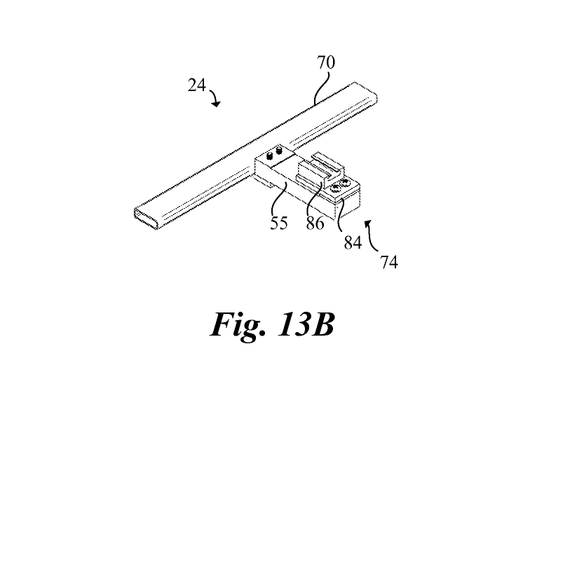

FIG. 13, which includes FIGS. 13A and 13B, show the internal components of measurement system 20 more clearly. In FIG. 13B, support structures 74 include a plate 84 and a rail attachment 86. Plate 84 is secured to load cell 55 using fasteners, welds, or other suitable devices. Rail attachment 86 is fixed to the upper surface of plate 84 and is configured such that slide rail 72 fits within its opening. This entire unit moves along slide rail 72 to adjust measurement device 24 to the desired position. Moreover, this entire unit may be taken out of measurement system 20 and replaced with a different configuration or measurement device.

In various embodiments, measurement device 24 is configured to minimize the total thickness of measurement system 20. For example, the thickness of measurement system 20 may be two and one-half inches in some examples. In other examples, the thickness of measurement system 20 may be even smaller.

As depicted in FIGS. 13A and 13B, slide rail 72 is configured to be smooth gliding, but resisting free movement under load to avoid lateral movement of strap 28 during testing. Moreover, the resistance of free movement by slide rail 72 may help avoid load cell or track damage when moving or adjusting bench incline angle.

Although not shown in FIG. 13, in some examples, slide rail 72 may include a locking mechanism along the rail. Slide rail 72 also may include a magnetic thin metal piece to assist with proper strap alignment.

In still other examples, an indicator may be present on strap 28. This indicator may be a brightly colored thin strip of tape in the middle of strap 28 to help align strap 28 on the subject quickly, with minimal or no assistance. Strap 28 may be further configured to include a snap on/off connector or adjustable pressure fit metal feed through strap connector.

In yet another embodiment, the strap may be adjusted relative to a spine of the subject to test force exerted against the body contact device by other muscles. For example, openings 78 and 80 may span almost the entire side surface of the metal housing. In this instance, measurement device 24 may be adjusted such that strap 28 bisects the pectoral muscles of subject 14.

Alternatively, strap 28 may be replaced by a body contact device having two handles, one protruding from each opening 78 and 80 and connected to load cell 55. Subject 14 may then exert force against the handles using a push motion. Measurement system 20 operates in the same manner as described previously to determine a strength measurement for subject 14, namely, the strength of the subject's pectoralis major, pectoralis minor, and auxiliary muscles.

In this manner, various muscle groups may be tested for strength, imbalance, and flexibility. Thus, measurement device 24 may be adjusted or aligned in different ways to accurately measure strength of various muscle groups.

Embodiments of the present invention include an apparatus including a back plate having an upper surface and a lower surface, a body contact device connected to the back plate, and a measurement device connected to the body contact device. The body contact device is configured to resist movement in a direction substantially perpendicular to the upper surface of the back plate. The body contact device may include a body strap having a first end and a second end, the first end being connected to a first side of the load cell and the second end being connected to a second side of the load cell. The measurement device is configured to measure a force applied to the body contact device. The measurement device may include a load cell. In some embodiments, padded layer is arranged along the upper surface of the back plate.

Furthermore, in some embodiments, a seating element is connected to the back plate.

In other embodiments, the back plate includes a metal housing having a top face, a bottom face, and two side faces connecting the top face and the bottom face. The measurement device is arranged within the interior of the metal housing. In some embodiments, a slide rail couples the measurement device and the back plate such that measurement device is movably connected to the slide rail. The measurement device is configured to move along a longitudinal axis of the back plate. In still other embodiments, fasteners are connected to the back plate. These fasteners may be bench belts that are configured to couple the back plate to a surface such as a bench.

Further, embodiments of the present invention include an abdominal strength measurement system that includes a metal housing, a load cell, and a body strap. The metal housing has a top face, a bottom face, and two side faces connecting the top face and the bottom face. The load cell is arranged within an interior of the metal housing. The body strap is connected to the load cell and is configured to resist movement in a direction substantially perpendicular to the top face of the metal housing.

In some embodiments, the two side faces of the metal housing include openings. A slide rail couples the load cell and the metal housing while an elongate member is connected to the load cell. A first end of the elongate member extends through an opening in a first side face of the metal housing and a second end of the elongate member extends through an opening in a second side face of the metal housing opposite the first side face. In other embodiments, the metal housing may be one contiguous metal plate.

In various embodiments, the abdominal strength measurement system includes an adjustable bench having a seat and a back plate. The back plate is configured to be adjusted to an angle of no more than 45 degrees to horizontal with the metal housing fixed to the back plate. In some embodiments, the back plate is adjusted to an angle of substantially 30 degrees to the horizontal. In still other embodiments, the abdominal strength measurement system includes a display device.

A method for measuring strength is also presented. An apparatus includes a back plate having an upper surface and a lower surface is provided. A force exerted against a body contact device connected to the back plate is measured. The force is applied in a direction perpendicular to the upper surface of the back plate. A strength measurement is determined from the force exerted against the body contact device.

In some embodiments, the strength measurement is displayed on a display device. The strength measurement is compared to a known strength scale. The strength measurement is then classified based on the comparison of the strength measurement to the known strength scale. Various embodiments further include determining an abdominal strength from the strength measurement. With these embodiments, the body contact device is arranged on a xiphoid process of a person such that the body contact device lies perpendicular to the xiphoid process.

In some embodiments, a report is generated for the subject. This report may include a strength score, the results of the comparison, and information about the subject's classification. The report may then be sent to the subject through a wireless communication system. The report also may be printed. The report may then be stored in a data storage device.

In still other embodiments, a method for measuring back strength is presented. An apparatus including a back plate may be provided. The back plate may be adjusted to a desired angle from the horizontal. A subject may be arranged on the apparatus such that a chest of the subject faces the back plate. A force exerted on a body contact device connected to the back plate is then measured. This force may be exerted by muscles of the posterior portion of the subject's torso. A strength measurement is then determined from the force exerted against the body contact device by the posterior torso muscles of the subject.

In some embodiments, the body contact device may be a strap. The strap may be adjusted to different positions along the posterior torso of the subject to measure strength of different muscle groups. Further, the apparatus may be configured to test both abdominal strength and lumbar extension at substantially the same time.

Advantages of the embodied devices include the ability to obtain an accurate abdominal strength measurement. In some embodiments, the apparatus is designed such that maximal force from the rectus abdominis is measured without the risk of assistance from non-abdominal musculature, gravity, or other devices. With an accurate abdominal strength measurement, various health characteristics of an individual may be assessed, including, but not limited to, a risk of obesity, lower back pain, back injury, and muscle imbalances, among others. Various embodiments described herein also provide a mobile, light-weight measurement device that is suitable for a variety of applications.

Moreover, with the use of an illustrative embodiment, clinicians can track abdominal strength of an individual over time. The apparatus provides accurate, repeatable testing of abdominal strength. As a result, clinicians may prescribe treatment or design training protocols based on the results of the abdominal strength measurements.

While this invention has been described with reference to illustrative embodiments, this description is not intended to be construed in a limiting sense. Various modifications and combinations of the illustrative embodiments, as well as other embodiments of the invention, will be apparent to persons skilled in the art upon reference to the description. As an illustration, the embodiments described in FIG. 1-13 may be combined with each other in various embodiments. It is therefore intended that the appended claims encompass any such modifications or embodiments.

Although the present invention and its advantages have been described in detail, it should be understood that various changes, substitutions and alterations can be made herein without departing from the spirit and scope of the invention as defined by the appended claims. For example, it will be readily understood by those skilled in the art that many of the features, functions, processes, and materials described herein may be varied while remaining within the scope of the present invention.

What is claimed is:

1. An apparatus comprising:
a back plate having an upper surface and a lower surface;
a body contact device connected to the back plate and configured to resist movement in a direction substantially perpendicular to the upper surface of the back plate; and
a measurement device connected to the body contact device and configured to measure a force applied to the body contact device.

2. The apparatus of claim 1, further comprising a padded layer arranged along the upper surface of the back plate.

3. The apparatus of claim 1, further comprising a seating element connected to the back plate.

4. The apparatus of claim 1, wherein the measurement device comprises a load cell.

5. The apparatus of claim 4, wherein the body contact device comprises a body strap having a first end and a second end, the first end being connected to a first side of the load cell and the second end being connected to a second side of the load cell.

6. The apparatus of claim 1, wherein the back plate comprises a metal housing having a top face, a bottom face, and two side faces connecting the top face and the bottom face and wherein the measurement device is arranged within an interior of the metal housing.

7. The apparatus of claim 1, further comprising a slide rail coupling the measurement device and the back plate, wherein the measurement device is movably connected to the slide rail and configured to move along a longitudinal axis of the back plate.

8. The apparatus of claim 1, further comprising fasteners connected to the back plate and configured to couple the back plate to a surface.

9. The apparatus of claim 8, wherein the fasteners comprise bench belts configured to couple the back plate to a bench.

10. An abdominal strength measurement system comprising:
a metal housing having a top face, a bottom face, and two side faces connecting the top face and the bottom face;
a load cell arranged within an interior of the metal housing; and
a body strap connected to the load cell, wherein the body strap is configured to resist movement in a direction substantially perpendicular to the top face of the metal housing.

11. The abdominal strength measurement system of claim 10, further comprising an adjustable bench having a seat and a back plate, wherein the back plate is configured to be adjusted to an angle of no more than 45 degrees to horizontal, and wherein the metal housing is fixed to the back plate.

12. The abdominal strength measurement system of claim 11, wherein the back plate is adjusted to an angle of substantially 30 degrees to the horizontal.

13. The abdominal strength measurement system of claim 10, wherein the two side faces of the metal housing comprise openings, and the abdominal strength measurement system further comprises:
a slide rail coupling the load cell and the metal housing; and
an elongate member connected to the load cell, wherein a first end of the elongate member extends through an opening in a first side face of the metal housing and a second end of the elongate member extends through an opening in a second side face of the metal housing opposite the first side face.

14. The abdominal strength measurement system of claim 10, wherein the metal housing comprises one contiguous metal plate.

15. The abdominal strength measurement system of claim 10, further comprising a display device.

16. A method for measuring strength comprising:
providing an apparatus comprising a back plate having an upper surface and a lower surface;
measuring a force exerted against a body contact device connected to the back plate in a direction perpendicular to the upper surface of the back plate; and
determining a strength measurement from the force exerted against the body contact device.

17. The method of claim 16, further comprising displaying the strength measurement on a display device.

18. The method of claim 17, further comprising:
comparing the strength measurement to a known strength scale; and
classifying the strength measurement based on the comparing the strength measurement to the known strength scale.

19. The method of claim 17, further comprising determining an abdominal strength from the strength measurement.

20. The method of claim 19, further comprising arranging the body contact device on a xiphoid process of a person such that the body contact device lies perpendicular to the xiphoid process.
